(19) United States
(12) Patent Application Publication
(10) Pub. No.: US 2017/0296069 A1
(43) Pub. Date: Oct. 19, 2017

(54) VAGINAL BLOOD-INSPECTING APPARATUS

(71) Applicants: ELIAS OREN, HERZLIYA (IL); ELIOR ITZKOVITZ COHEN, NETANYA (IL)

(72) Inventors: ELIAS OREN, HERZLIYA (IL); ELIOR ITZKOVITZ COHEN, NETANYA (IL)

(21) Appl. No.: 15/486,320

(22) Filed: Apr. 13, 2017

(30) Foreign Application Priority Data
Apr. 19, 2016 (IL) 245218

(51) Int. Cl. A61B 5/02; A61B 5/00

(52) U.S. Cl. CPC A61B 5/02042; A61B 5/4337

(57) ABSTRACT

A vaginal blood-inspecting apparatus, including: a cloth, including a sheet, a rod, such that the first end thereof is for being wrapped by the sheet of the cloth, and the second end thereof is for pushing thereon from outside the vagina, for inserting the cloth thereinto, and a hollow cylinder, for covering a portion of the rod and of the cloth, thereby the hollow cylinder maintains the wrapping of the sheet on the rod, thereby sliding of the hollow cylinder towards the second end of the rod removes the hollow cylinder from the rod without removing the sheet from the rod, thereby exposing the cloth to the internal wall of the vagina.

### CROSS-REFERENCE TO RELATED APPLICATIONS

This application claims the benefit of priority from Israel Patent No. 245218, filed Apr. 19, 2016, the disclosure of which is incorporated herein by reference.

### TECHNICAL FIELD

The invention relates to the field of ritual purity in Judaism. More particularly, the invention relates to a vaginal blood-inspecting apparatus therefor.

### BACKGROUND

Jewish women inspect presence of blood in the vagina by inserting a white cloth.

However, the white cloth might hurt the walls of the vagina during the insertion.

US2010286552 discloses a vaginal blood-inspecting apparatus, including an inspecting cloth.

### BRIEF DESCRIPTION OF THE DRAWINGS

Embodiments, features, and aspects of the invention are described herein in conjunction with the following drawings:

FIG. 1 depicts the first step of using the vaginal blood-inspecting apparatus, according to one embodiment of the invention.

FIG. 2 depicts the second step of using the vaginal blood-inspecting apparatus of FIG. 1.

FIG. 3 depicts the state upon applying the second step.

FIG. 4 depicts two parts constituting the vaginal blood-inspecting apparatus of FIG. 1.

FIG. 5 depicts the third step of using the vaginal blood-inspecting apparatus of FIG. 1.

FIG. 6 depicts the fourth step of using the vaginal blood-inspecting apparatus of FIG. 1.

FIG. 7 depicts the fifth step and the optional sixth step of using the vaginal blood-inspecting apparatus of FIG. 1.

FIG. 8 is a perspective view of the cloth of FIG. 5 alone.

FIG. 9 depicts the sixth and the seventh steps of using the vaginal blood-inspecting apparatus of FIG. 1, according to a second optional use.

The drawings are not necessarily drawn to scale.

### DETAILED DESCRIPTION

The invention will be understood from the following detailed description of embodiments of the invention, which are meant to be descriptive and not limiting. For the sake of brevity, some well-known features, methods, systems, procedures, components, circuits, and so on, are not described in detail.

The reference numbers have been used to point out elements in the embodiments described and illustrated herein, in order to facilitate the understanding of the invention. They are meant to be merely illustrative, and not limiting. Also, the foregoing embodiments of the invention have been described and illustrated in conjunction with systems and methods thereof, which are meant to be merely illustrative, and not limiting.

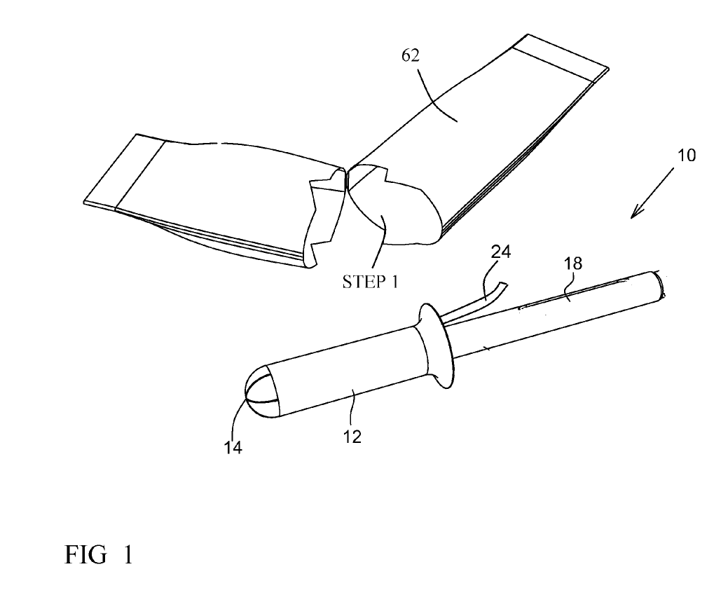

FIG. 1 depicts the first step of using the vaginal blood-inspecting apparatus, according to one embodiment of the invention.

At the first step, the user cuts the packaging 62 and reveals the vaginal blood-inspecting apparatus 10, according to one embodiment of the invention.

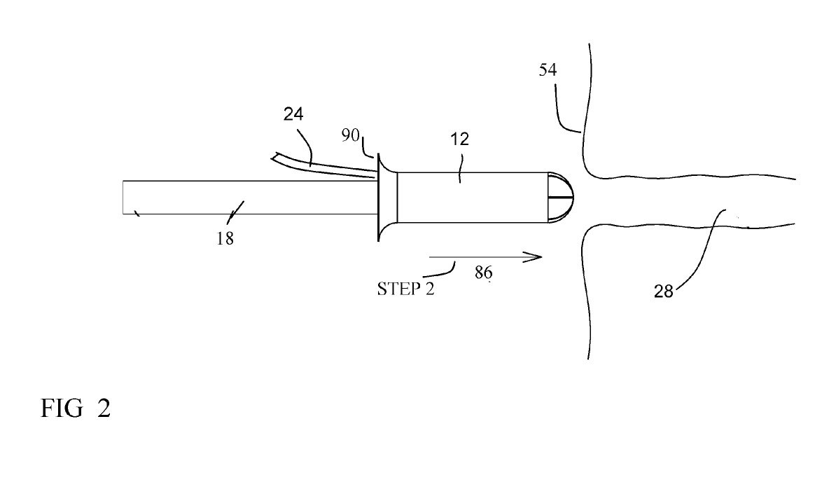

FIG. 2 depicts the second step of using the vaginal blood-inspecting apparatus of FIG. 1.

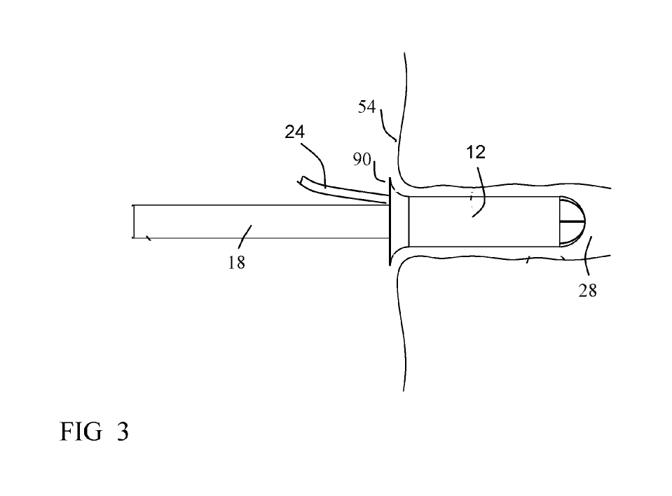

FIG. 3 depicts the state upon applying the second step.

At the second step, the user pushes a rod 18 of vaginal blood-inspecting apparatus 10 into the vagina 28, as depicted by arrow 86, for inserting a cloth 24 into the vagina 28.

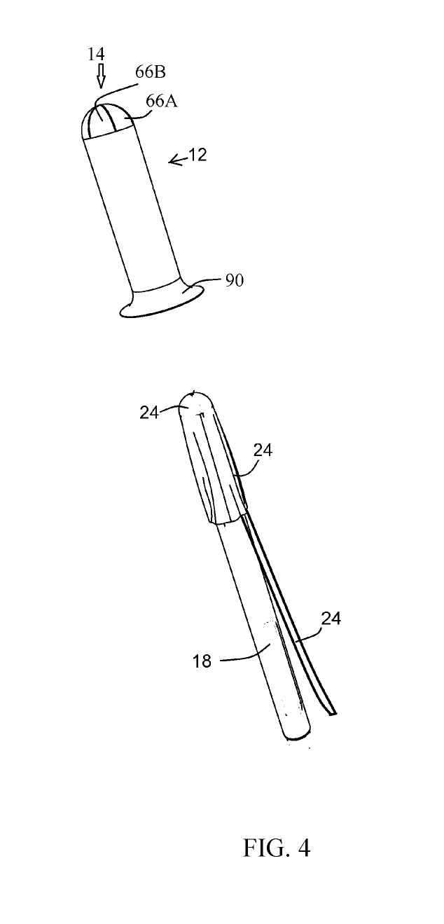

FIG. 4 depicts two parts constituting the vaginal blood-inspecting apparatus of FIG. 1.

Vaginal blood-inspecting apparatus 10 includes a rod 18; a cloth 24 surrounding rod 18; and a hollow cylinder 12, for covering a portion of rod 18 and of cloth 24. Except for hollow cylinder 12, connecting cloth 24 to rod 18 by said covering, cloth 24 is not connected to rod 18 by any other means.

Hollow cylinder 12 is typically made of plastic for separating cloth 24 from the internal wall 94 (indicated in FIG. 3) of vagina 28 during the insertion of cloth 24 into vagina 28, for avoiding cloth 24 rub and hurt the vagina wall 94.

Hollow cylinder 12 includes at the end thereof, a hole covered by a peripheral elastic lips element 14, including lips 66A, 66B, etc.

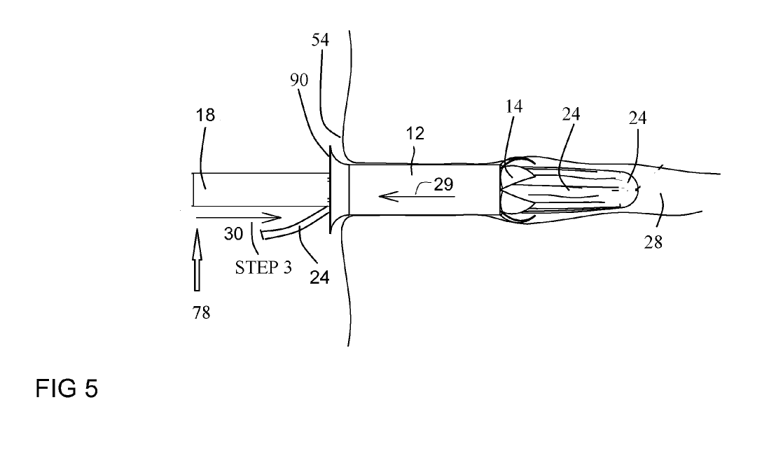

FIG. 5 depicts the third step of using the vaginal blood-inspecting apparatus of FIG. 1.

At the third step, the user further pushes rod 18, as depicted by arrow 30, for further inserting rod 18 and cloth 24 into vagina 28,

Either a broadening zone 90 of hollow cylinder 12, or the finger's user do not allow hollow cylinder 12 to further enter vagina 28. Consequently, rod 18 and cloth 24 slide together in relation to hollow cylinder 12, by opening peripheral elastic lips element 14 of hollow cylinder 12, thereby revealing cloth 24 to vagina 28.

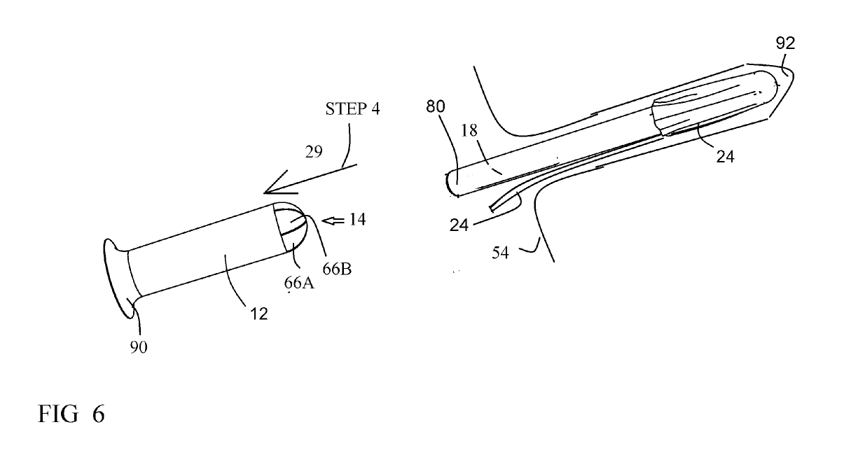

FIG. 6 depicts the fourth step of using the vaginal blood-inspecting apparatus of FIG. 1.

At the fourth step, the user pulls hollow cylinder 12, as depicted by arrow 29, and may further push rod 18 for making cloth 24 reach even the deepest end 92 of vagina 28.

The user may, instead, not introduce hollow cylinder 12 into vagina 28, and may only push rod 18 only, thereby only rod 18 and cloth 24 enter vagina 28, such that rod 18 and cloth 24 slide in relation to hollow cylinder 12, by opening peripheral elastic lips element 14, thereby revealing cloth 24 to vagina 28.

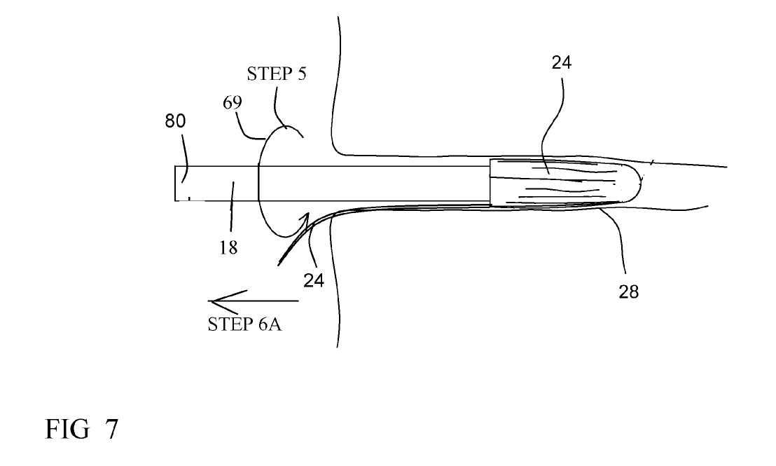

FIG. 7 depicts the fifth step and the optional sixth step of using the vaginal blood-inspecting apparatus of FIG. 1.

At the fifth step, the user may move rod 18 in relation to vagina 28, as shown by arrow 69, for moving cloth 24 in relation to vagina 28, for receiving blood if present.

At the sixth step of a first optional use, denoted step 6A, the user may pull rod 18 together with cloth 24, out of vagina 28, for viewing cloth 24 regarding if it has received blood.

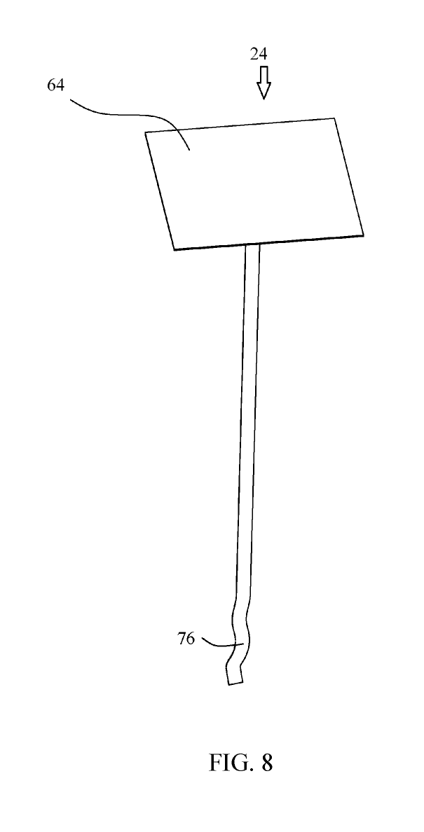

FIG. 8 is a perspective view of the cloth of FIG. 5 alone.

Cloth 24 includes a sheet 64 and a tail 76.

Thus, hollow cylinder 12 of FIG. 2 functions for wrapping sheet 64 cloth 24 around rod 18. This wrapping is sufficient for moving cloth 24 by rod 18, even after the removal of hollow cylinder 12.

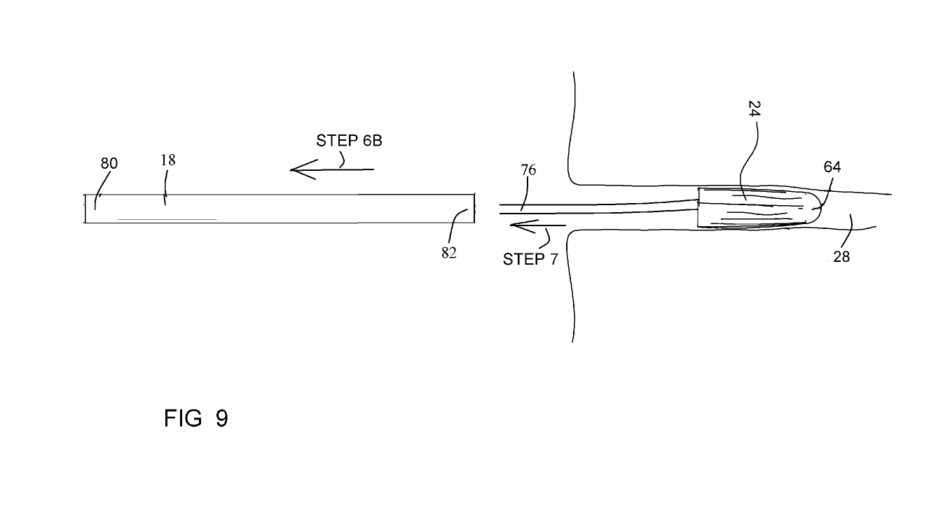

FIG. 9 depicts the sixth and the seventh steps of using the vaginal blood-inspecting apparatus of FIG. 1, according to a second optional use.

Optionally, at the sixth step, denoted step 6B, being a second option use, the user may pull rod 18 outside vagina 28, while leaving cloth 24 inside vagina 28.

At the ninth step, the user may pull tail 76 of cloth 24, for pulling the entire cloth 24 outside, for viewing cloth 24 regarding if it has received blood during the long period.

Thus, in one aspect, the invention is directed to a vaginal blood-inspecting apparatus (10), including:
a cloth (24), including a sheet (64);
a rod (18), such that the first end (82) thereof is for being wrapped by the sheet (64) of the cloth (24), and the second end (80) thereof is for pushing thereon from outside (78) the vagina (28), for inserting the cloth (24) thereinto; and
a hollow cylinder (12), for covering a portion of the rod (18) and of the cloth (24), thereby the hollow cylinder (12) maintains the wrapping of the sheet (64) on the rod (18),
thereby sliding of the hollow cylinder (12) towards the second end (80) of the rod (18) removes the hollow cylinder (12) from the rod (18) without removing the sheet (64) from the rod (18), thereby exposing the cloth (24) to the internal wall (94) of the vagina (28),
thereby, once the cloth (24) is exposed, moving of the first end (82) of the rod (18) within the vagina (28) by moving the second end (80) of the rod (18), rubs the cloth (24) against the internal wall (94) of the vagina (28).

The hollow cylinder (12) may include a peripheral elastic lips element (14) disposed at one end thereof, for providing peripheral pressing of the sheet (64) onto the first end (82) of the rod (18),
thereby the peripheral elastic lips element (14) maintains the wrapping of the sheet (64) on the rod (18) upon the sliding of the hollow cylinder (12) towards the second end (80) of the rod (18).

The cloth (24) may further include at least one tail (76) extending from one end of the sheet (64), wherein the at least one tail is sufficiently long for extending outside (78) the vagina (28),
thereby holding of the tail (76) and of the rod (18) together by fingers, both from outside (78) the vagina (28), allows maintaining the wrapping the wrapping of the sheet (64) on the rod (18) even while pulling the rod (18).

In the figures and/or description herein, the following reference numerals (Reference Signs List) have been mentioned:
numeral 10 denotes the vaginal blood-inspecting apparatus, according to one embodiment of the invention;
numeral 12 denotes the hollow cylinder;
numeral 14 denotes a peripheral elastic lips element of the hollow cylinder, for allowing threading the cloth therethrough;
numeral 18 denotes a rod for holding the cloth;
numeral 24 denotes the cloth;
numeral 28 denotes the vagina;
numeral 29 denotes an arrow;
numeral 54 denotes the entrance wall of the vagina;
numeral 62 denotes a packaging;
numerals 66A and 66B denote lips of the lips element of the hollow cylinder;
numerals 69 denotes an arrows
numeral 78 denotes working area for the user's fingers outside the vagina;
numeral 80 denotes the external end of the rod;
numeral 82 denotes the internal end of the rod;
numeral 86 denotes an arrow;
numeral 90 denotes the broadening zone of the hollow cylinder;
numeral 92 denotes the deepest end of the vagina;
numeral 94 denotes the internal wall of the vagina;

The foregoing description and illustrations of the embodiments of the invention has been presented for the purposes of illustration. It is not intended to be exhaustive or to limit the invention to the above description in any form.

Any term that has been defined above and used in the claims, should to be interpreted according to this definition.

The reference numbers in the claims are not a part of the claims, but rather used for facilitating the reading thereof. These reference numbers should not be interpreted as limiting the claims in any form.

What is claimed is:

1. A vaginal blood-inspecting apparatus, comprising:
a cloth, comprising a sheet;
a rod, a first end thereof for being wrapped by said sheet of said cloth, and a second end thereof for pushing thereon from outside a vagina, for inserting said cloth thereinto; and
a hollow cylinder, for covering a portion of said rod and of said cloth, thereby said hollow cylinder maintains said wrapping of said sheet on said rod,
thereby sliding of said hollow cylinder towards said second end of said rod allows removing said hollow cylinder from said rod without removing said sheet from said rod, thereby exposing said cloth to an internal wall of the vagina,
thereby, once said cloth is exposed, moving of said first end of said rod within the vagina by moving said second end of said rod, rubs said cloth against said internal wall of the vagina.

2. A vaginal blood-inspecting apparatus according to claim 1, wherein said hollow cylinder comprises a peripheral elastic lips element disposed at one end thereof, for providing peripheral pressing of said sheet onto said first end of said rod,
thereby said peripheral elastic lips element maintains said wrapping of said sheet on said rod upon said sliding of said hollow cylinder towards said second end of said rod.

3. A vaginal blood-inspecting apparatus according to claim 1, wherein said cloth further comprises at least one tail extending from an end of said sheet, said at least one tail sufficiently long for extending outside the vagina,
thereby holding of said tail and of said rod together, both from outside the vagina, allows maintaining said wrapping said wrapping of said sheet on said rod even while pulling said rod.
